SEQUENCE LISTING (11 sequences)

SEQ ID NO: 1 - DNA, 33 bp, Artificial Sequence
  Description of Artificial Sequenceluciferase-specific hairpin siRNA
ggattccaattcagcgggagccacctgatggaa

SEQ ID NO: 2 - DNA, 35 bp, Artificial Sequence
  Description of Artificial Sequenceluciferase-specific hairpin siRNA
ttcgatcaggtggctcccgctgaattggaatcctt

SEQ ID NO: 3 - DNA, 28 bp, Artificial Sequence
  Description of Artificial Sequencebeta3-integrin-specific hairpin s1RNA
gaactattagagctgcctgtgcctgaga

SEQ ID NO: 4 - DNA, 30 bp, Artificial Sequence
  Description of Artificial Sequencebeta3-integrin-specific hairpin s1RNA
tctgaggcacaggcagctctaatagttctt

SEQ ID NO: 5 - DNA, 27 bp, Artificial Sequence
  Description of Artificial Sequencebeta3-integrin-specific hairpin s1RNA
gaactattagagctgcctgtgcctcgt

SEQ ID NO: 6 - DNA, 29 bp, Artificial Sequence
  Description of Artificial Sequencebeta3-integrin-specific hairpin s1RNA
tgcaggcacaggcagctctaatagttctt

SEQ ID NO: 7 - DNA, 15 bp, Artificial Sequence
  Description of Artificial Sequenceportion of sequence inserted into PCR primer
ccaaacgcgtaaaan

SEQ ID NO: 8 - DNA, 22 bp, Artificial Sequence
  Description of Artificial Sequenceportion of sequence inserted into PCR primer
ngtgtttcgtcctttccacaag

SEQ ID NO: 9 - DNA, 93 bp, Artificial Sequence
  Description of Artificial Sequencehp-RNA primer
ccaaacgcgtaaaaagaactattagagctgcctgtgcctcaagcttcaggcacaggcagctctaatagttcggtgtttcgtcctttccacaag

SEQ ID NO: 10 - DNA, 24 bp, Artificial Sequence
  Description of Artificial Sequencesecond primer US-F
cagaggaacaggtcgaccaaggtc

SEQ ID NO: 11 - DNA, 18 bp, Artificial Sequence
  Description of Artificial SequenceU6-F primer
ggactatcatatgcttac

(19) United States
(12) Patent Application Publication
(10) Pub. No.: US 2004/0005593 A1
(43) Pub. Date: Jan. 8, 2004

(54) Novel method for delivery and intracellular synthesis of siRNA molecules

(75) Inventor: James Lorens, Portola Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Rigel Pharmaceuticals, Inc., South San Francisco, CA (US)

(21) Appl. No.: 10/383,835

(22) Filed: Mar. 6, 2003

Related U.S. Application Data
(60) Provisional application No. 60/380,567, filed on May 13, 2002.
(60) Provisional application No. 60/362,468, filed on Mar. 6, 2002.

(51) Int. Cl. C12Q 1/68; G01N 33/53; C07H 21/04; C12P 21/02; C12N 5/06; C07K 14/435; C07K 14/16

(52) U.S. Cl. 435/6; 435/7.1; 435/69.1; 435/320.1; 435/325; 530/350; 536/23.1

(57) ABSTRACT

The present invention relates to methods of screening for target polypeptides that bind to RNA, using affinity purification methods, and the use of such target polypeptide for drug discovery and in methods of treating and preventing disease.

### CROSS-REFERENCES TO RELATED APPLICATIONS

The present application claims priority to U.S. Ser. No. 60/362,468, filed Mar. 6, 2002, and U.S. Ser. No. 60/380,567, filed May 13, 2002, herein each incorporated by reference in their entirety.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Suppression of the expression of particular genes is an important tool both for target validation and for the identification of therapeutic agents for treatment of disease. Gene silencing can be accomplished by the introduction of a transgene corresponding to the gene of interest in the antisense orientation relative to its promoter (see, e.g., Sheehy et al., Proc. Nat'l Acad. Sci. USA 85:8805-8808 (1988); Smith et al., Nature 334:724-726 (1988)), or in the sense orientation relative to its promoter (Napoli et al., Plant Cell 2:279-289 (1990); van der Krol et al., Plant Cell 2:291-299 (1990); U.S. Pat. No. 5,034,323; U.S. Pat. No. 5,231,020; and U.S. Pat. No. 5,283,184), both of which lead to reduced expression of the transgene as well as the endogenous gene.

Posttranscriptional gene silencing or RNA interference (RNAi) has been reported to be accompanied by the accumulation of small (20-25, e.g., 20, 21, 22 nucleotide) fragments of double stranded RNA, which are reported to be synthesized from an RNA template (Hamilton & Baulcombe, Science 286:950-952 (1999)). These fragments are called small interfering RNAs (siRNAs). It has become clear that in a range of organisms, including mammals, siRNA is an important component leading to gene silencing (Fire et al., Nature 391:806-811 (1998); Timmons & Fire, Nature 395:854 (1998); WO99/32619; Kennerdell & Carthew, Cell 95:1017-1026 (1998); Ngo et al., Proc. Nat'l Acad. Sci. USA 95:14687-14692 (1998); Waterhouse et al., Proc. Nat'l Acad. Sci. USA 95:13959-13964 (1998); WO99/53050; Cogoni & Macino, Nature 399:166-169 (1999); Lohmann et al., Dev. Biol. 214:211-214 (1999); Sanchez-Alvarado & Newmark, Proc. Nat'l Acad. Sci. USA 96:5049-5054 (1999); Elbashir et al., Nature 411:494-297 (2001)). As gene silencing is a powerful tool for regulation of gene expression, both of endogenous genes and of transgenes, improved methods of gene silencing are desired.

### SUMMARY OF THE INVENTION

The present invention provides expression vectors encoding targeted siRNA molecules or randomized siRNA molecules from about 15-30 basepairs, often about 19-28 base pairs in length, often about 24-29 base pairs in length, the vectors comprising in sequence, a pol III promoter, a first siRNA encoding sequence, a linker, a second siRNA encoding sequence, and a transcription terminator. In one embodiment, the linker optionally comprises a self-cleaving ribozyme. In another embodiment, the linker comprises a sequence that encodes a U-turn RNA. In another embodiment, the linker is about 4-8 bases in length, or about 5-6 bases in length. In one embodiment, the vector is a retroviral vector. In another embodiment, the retroviral vector is a conditional expression vector, with conditional expression optionally conferred by the tet operator overlapping the pol III promoter. In one embodiment, the pol III promoter is the U6 RNA promoter. In one embodiment, the vector comprises a marker for viral infection, e.g., a nucleic acid encoding a GFP. FIGS. 1 and 3 provide examples of the vectors of the invention.

The invention also provides siRNA libraries, methods of inhibiting expression of a target gene, and methods of determining the function of a gene. Preferably, the siRNA molecules are 19, 20, 21, 22, 23, 24, 25, 26, 27, 28, or 29 nucleotides in length.

### BRIEF DESCRIPTION OF THE DRAWINGS

FIG. 1 shows an expression vector of the invention encoding an siRNA.

FIG. 2 shows a method of making a library of vectors encoding randomized siRNAs.

FIG. 3 shows a conditional expression vector of the invention encoding an siRNA.

FIGS. 4 and 5 show that a retrovirally expressed β3-integrin specific hairpin siRNA stably reduces surface αv β3 levels.

### DETAILED DESCRIPTION OF THE INVENTION

Introduction

The present invention provides vectors and methods for making siRNA molecules, and the generation of randomized siRNA libraries.

The siRNA expression vectors of the invention are expressed in the cell or organism of choice, e.g., a bacterial cell, a fungal cell, a eukaryotic cell, e.g., a plant cell or a mammalian cell. In one embodiment, the siRNA expression vector is expressed in a mammalian cell for silencing of a target mammalian or viral gene. In another embodiment, the randomized siRNA expression vectors are used in functional genomics to determine the effect of regulating gene expression of a selected endogenous gene, exogenous gene, viral gene, or transgene.

In one embodiment, the siRNA expression vectors are retroviral expression vectors (see, e.g., Lorens et al., Curr. Opin. Biotechnol. 12:613-621 (2001)).

Suitable pol III promoters include ribosomal 5S RNA promoter, a U6 RNA promoter and promoters from other snRNAs, tRNA promoters, a 7SL promoter, adenoviral VA RNA promoters, and Epstein-Barr virus EBER RNA promoters.

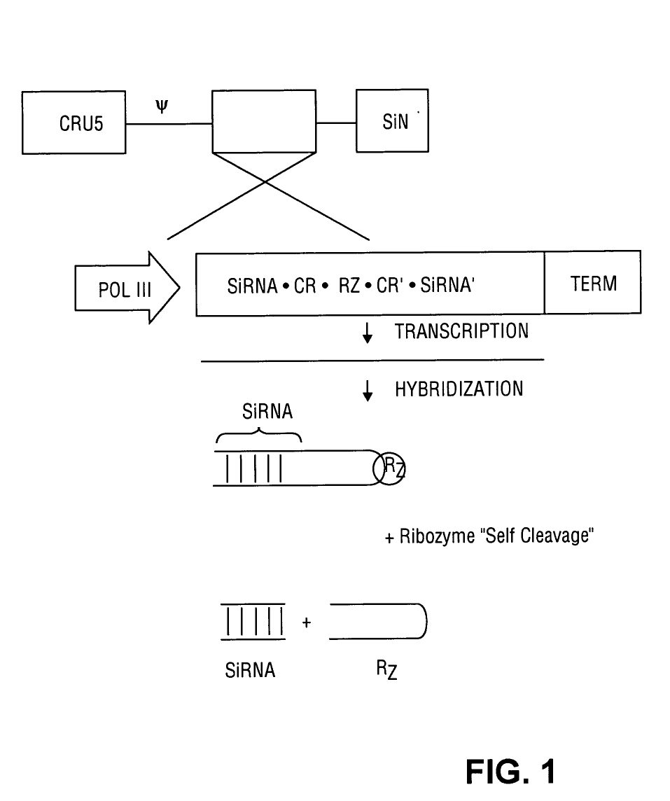

Suitable self splicing or self cleaving ribozymes of the invention include those having characteristics of group I intron ribozymes (see, e.g., Cech, 1995, Biotechnology 13:323), the characteristics of group II intron ribozymes (see, e.g., Swisher et al., J. Mol. Biol. 315:297-310 (2002), and the characteristics of hammerhead ribozymes (see, e.g., Edgington, 1992, Biotechnology 10:256). Methods of making and using ribozymes are known to those of skill in the art (see, e.g., Kuimelis & McLaughlin, Chem. Rev. 98:1027-1044 (1998); Zhou & Taira, Chem. Rev. 98:991-1026 (1998); Barroso-DelJesus & Berzal-Herranz, EMBO Rep. 2:1112-118 (2001); and Ciesiolka et al., Acta Biochim. Pol. 48:409-418 (2001)). In one embodiment, the ribozyme is a Tetrahymena rRNA intron ribozyme or a Neurospora VS ribozyme. FIG. 1 provides an example of an siRNA expression vector that includes a self-splicing ribozyme.

Linker RNAs having a U-turn motif are known to those of skill in the art (see, e.g., Zhang et al., Biochemistry 21:40 (2001); Sundaram et al., Biochemistry 39:15652 (2000); Hermann et al., Eur Biophys. J. 27:153-165 (1998); and Gutell et al., J. Mol. Biol. 300:791-803 (2000)). For example, a U turn RNA is found in a pol III promoter. Linkers can be 5-10 nucleotides in length, often 4, 5, 6, 7, 8, 9, or 10 nucleotides in length, or may be longer, e.g., 5-50 nucleotides in length (see, e.g., Brummelkamp et al., Sciencexpress, Mar. 21, 2002).

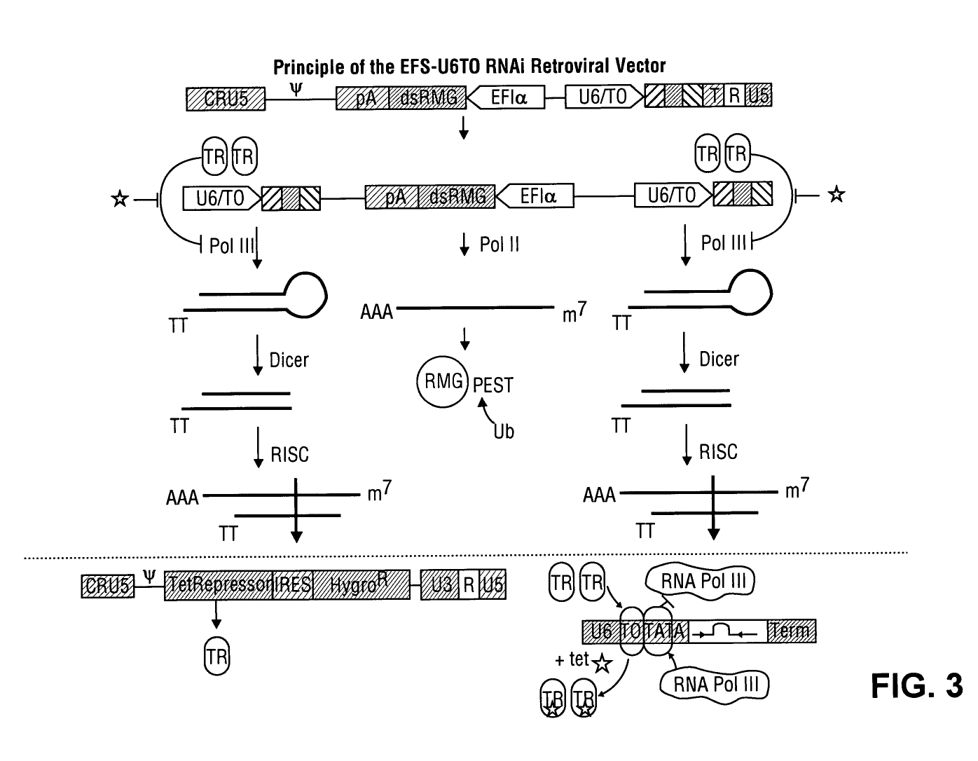

Optionally, the vector conditionally expresses the siRNA, e.g., using a tet operator linked to the pol III promoter (see Example I and FIG. 3). Conditional expression small molecule systems are typified by the tet-regulated systems, the RU-486 system, the ecdysone-regulated system, and a system incorporating a chimeric factor including a mutant progesterone receptor (see, e.g., Gossen & Bujard, Proc. Natl. Acad. Sci. U.S.A. 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the zinc finger protein activators and repressors and thus impart small molecule control on the target gene(s) of interest.

Suitable target genes include those associated with lymphocyte activation, angiogenesis, apoptosis, cellular proliferation, mast cell degranulation, viral replication, and viral translation. Phenotype assays for gene associated with lymphocyte activation, angiogenesis, apoptosis, cellular proliferation, mast cell degranulation, viral replication, and viral translation are well known to those of skill in the art.

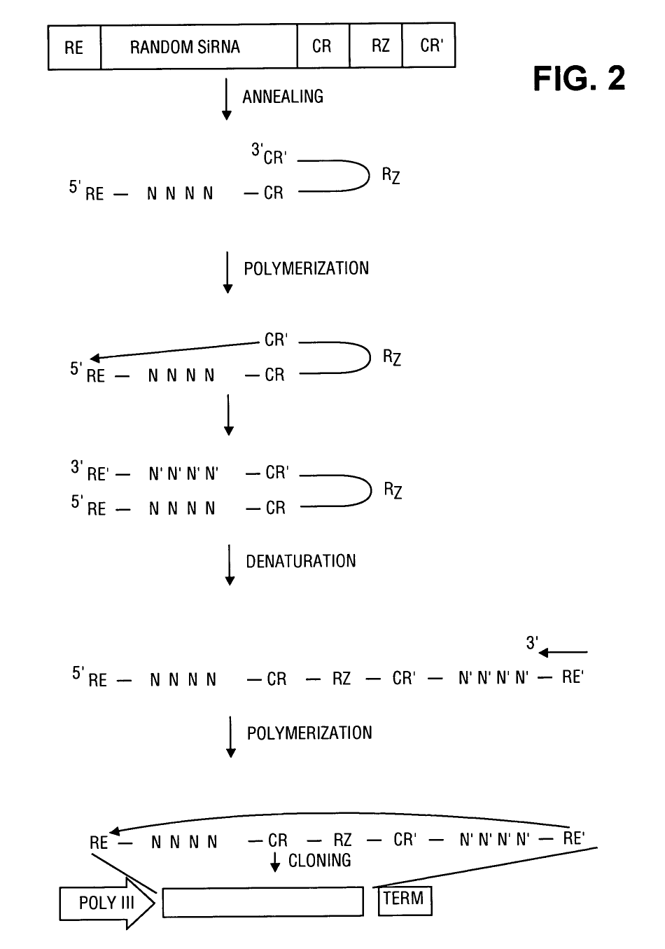

Random libraries of interfering RNA molecules may be constructed by synthesizing a pool of oligonucleotides comprising a restriction site, a randomized siRNA sequence, a complementarity region sequence, and a hairpin-forming linker sequence (optionally a U-turn motif, a ribozyme and/or or a two complementary sequences that form a hairpin or stem loop structure). The oligonucleotides will adopt a hairpin structure as shown in FIG. 2. This structure is a substrate for a DNA polymerase, facilitating the synthesis of a complement sequence of the randomized siRNA sequence. The hairpin structure is then denatured and hybridized to a primer at the 3′ end allowing the conversion of the total sequence to double stranded DNA by a DNA polymerase. The double stranded oligonucleotides encoding a random assortment of siRNA sequences are cloned into the retroviral vector described herein to generate an siRNA-expression vector library.

In order to enrich the libraries for siRNA molecules that correspond to expressed genes, the pool of oligonucleotides may first be hybridized to cDNA or RNA, and the binding oligonucleotides then cloned into the siRNA-expression vector library. Alternatively, a cDNA or RNA population may be fragmented or digested into fragments of about 15-30 nucleotides in length, and cloned into the siRNA expression vector library. In order to identify siRNA molecules that regulate a selected phenotype, specific cell types can be used as the source of cDNA or RNA, e.g., synchronized cells, cancer cells, lymphocytes, cells involved in angiogenesis, mast cell degranulation, virally infected cells, and cells undergoing apoptosis.

In another embodiment, the methods and libraries of the invention can be used to screen for siRNAs that efficiently regulate expression of a target gene. cDNA or RNA from the target gene can be used to make a library, and then the siRNA molecules of interest are selected by screening against cells expressing the target gene. Similarly, siRNAs that target selected domains, e.g., enzymatic domains, binding domains, etc. can be selected in the same manner. A cDNA or RNA from the target domain is used to make a library and then the siRNA molecules of interest are selected by screening against cells expressing the target domain, or against cells expressing a gene that includes the target domain.

Finally, the methods and expression vectors of the invention can be used to screen for modulators of a pathway by identifying siRNA molecules that regulate a single member of the pathway. Such methods can be used to look for activation as well as inhibition of the pathway.

Definitions

“Sequence encoding a self cleaving or self splicing ribozyme” refers to a ribozyme and flanking sequences that are cleaved by the ribozyme. A “self-cleaving or self splicing ribozyme” is a ribozyme that recognizes and cleaves flanking sequences, thus release the ribozyme from the flanking sequences.

“U-turn RNA” refers to an RNA sequence of at least 4-8, preferably at least 5-6 nucleotides that forms a loop structure.

A “target gene” refers to any gene suitable for regulation of expression, including both endogenous chromosomal genes and transgenes, as well as episomal or extrachromosomal genes, mitochondrial genes, chloroplastic genes, viral genes, bacterial genes, animal genes, plant genes, protozoal genes and fungal genes.

An “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. In another embodiment, a “randomized siRNA” refers to a nucleic acid that forms a double stranded siRNA, wherein the sequence of the siRNA is randomized. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

“Inverted repeat” refers to a nucleic acid sequence comprising a sense and an antisense element positioned so that they are able to form a double stranded siRNA when the repeat is transcribed. The inverted repeat may optionally include a linker or a heterologous sequence between the two elements of the repeat. The elements of the inverted repeat have a length sufficient to form a double stranded RNA. Typically, each element of the inverted repeat is about 15 to about 100 nucleotides in length, preferably about 20-30 base nucleotides, preferably about 20-25 or 24-29 nucleotides in length, e.g., 20, 21, 22, 23, 24, 25, 26, 27, 28, 29, or 30 nucleotides in length.

“Substantial identity” refers to a sequence that hybridizes to a reference sequence under stringent conditions, or to a sequence that has a specified percent identity over a specified region of a reference sequence.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization.

Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

The terms “substantially identical” or “substantial identity,” in the context of two or more nucleic acids or polypeptide sequences, refer to two or more sequences or subsequences that are the same or have a specified percentage of amino acid residues or nucleotides that are the same (i.e., at least about 60%, preferably 65%, 70%, 75%, preferably 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. Preferably, the substantial identity exists over a region that is at least about 6-7 amino acids or 25 nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The phrase “inhibiting expression of a target gene” refers to the ability of a siRNA of the invention to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, preferably 50%, more preferably 25-0%. Suitable assays include those described below in the Example section, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

A “label” or a “detectable moiety” is a composition detectable by spectroscopic, photochemical, biochemical, immunochemical, chemical, or other physical means. For example, useful labels include ³²p, fluorescent dyes, electron-dense reagents, enzymes (e.g., as commonly used in an ELISA), digoxigenin, biotin, luciferase, CAT, beta galactosidase, GFP, or haptens and proteins which can be made detectable, e.g., by incorporating a radiolabel into the peptide or used to detect antibodies specifically reactive with the peptide.

“Biological sample” includes tissue; cultured cells, e.g., primary cultures, explants, and transformed cells; cellular extracts, e.g., from cultured cells or tissue, cytoplasmic extracts, nuclear extracts; blood, etc. Biological samples include sections of tissues such as biopsy and autopsy samples, and frozen sections taken for histologic purposes. A biological sample, including cultured cells, is typically obtained from a eukaryotic organism, most preferably a mammal such as a primate, e.g., chimpanzee or human; cow; dog; cat; a rodent, e.g., guinea pig, rat, mouse; rabbit; or a bird; reptile; or fish.

“Nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation tumor cell proliferation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

Vector Synthesis

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

siRNAs and nucleic acids encoding siRNA expression vectors are constructed using methods well know to those of skill in the art. siRNAs that have substantial or complete identity to a target sequence can be cloned or synthesized according to methods well known to those of skill in the art. Randomized siRNA molecules are likewise made using methods known to those of skill in the art. In one embodiment, FIG. 1 shows an exemplary siRNA expression vector, comprising either a targeted or a randomized siRNA and a self-cleaving ribozyme. In another embodiment, the expression vector comprises a linker sequence that forms a U-turn RNA. FIG. 2 shows a method of making a randomized siRNA library.

Methods for making and screening cDNA libraries are well known (see, e.g., Gubler & Hoffman, Gene 25:263-269 (1983); Sambrook et al., supra; Ausubel et al., supra), as are PCR methods (see U.S. Pat. Nos. 4,683,195 and 4,683,202; PCR Protocols: A Guide to Methods and Applications (Innis et al., eds, 1990)). Expression libraries are also well known to those of skill in the art.

Expression in Prokaryotes and Eukaryotes

To obtain expression of an siRNA gene, one typically subclones the two complementary portions encoding the first and second siRNA sequence into an expression vector that contains a strong promoter to direct transcription, preferably a pol II promoter, a linker between the first and second siRNA sequences, and a transcription terminator. Bacterial expression systems are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Selection of the pol III promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. Suitable pol III promoters include ribosomal 5S RNA promoter, tRNA promoters, a7SL promoters, adenoviral VA RNA promoters, and Epstein-Barr virus EBER RNA promoters. In addition, the expression vector can comprise internal pol III control elements known to those of skill in the art.

In addition to the pol III promoter, the expression vector typically contains a transcription unit or expression cassette that contains all the additional elements required for the expression of the siRNA in host cells.

In addition to a promoter sequence, the expression cassette should also contain a transcription termination region downstream of the siRNA construct to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, and baculovirus pDSVE. In one embodiment, retroviral vectors are preferred.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983). Any of the well-known procedures for introducing foreign nucleotide sequences into host cells may be used. These include the use of viral transduction, calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least siRNA construct into the host cell.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following example is provided by way of illustration only and not by way of limitation. Those of skill in the art will readily recognize a variety of noncritical parameters that could be changed or modified to yield essentially similar results.

### Example 1

The EFS-U6TO Vector for Conditional Expression of siRNA

The EFS-U6TO vector is retroviral construct designed to stably and conditionally express short hairpin RNAs (hp-RNA) that can exert long term regulated RNA interference (RNAi) in mammalian cells (see FIG. 3). The EFS-U6TO vector comprises retroviral elements required for stable integration into the genome of infected cells, a modified U6 RNA promoter and terminator imbedded within the 3′LTR for conditional expression of hp-RNA and an internal EFI-α expression cassette driving a destabilized version (C-terminal PEST sequence) of the Renilla GFP (dsRMG) for independent monitoring of transfection/infection efficiencies.

Upon infection the 3′LTR-containing U6TO-hp-RNA expression cassette is duplicated to create the 5′LTR. This vector proviral form integrates stably into random regions of the target cell genome. The EF1-(α expression expresses dsRMG in a RNA pol II dependent manner and serves as a marker of viral infection. The C-terminal PEST sequence targets the GFP for ubiquitin-dependent proteolysis. This increases the turnover rate of the otherwise hyperstable GFP.

The LTRs containing modified U6 RNA promoters (U6TO) express short hp-RNAs in an RNA pol III dependent manner. A poly-T tract serves as a termination sequence. The EFS-U6TO is a self-inactivating (SIN) vector as the viral promoter/enhancer activity is lost upon integration. As the hp-RNA and GFP transcripts are discontinuous in the proviral form, there is no RNAi effect on the vector itself.

The U6TO is a composite type III RNA pol III promoter that comprises Pol III transcription factor recognition sites and a tet-operator sequence (TO) overlapping the TATA-box. The bacterial Tet repressor protein (TR) binds tightly to the tet-operator tightly leading to steric blockade of the pol III recognition sites and inhibition of transcription. TR is expressed from a second retroviral vector (CTRIH) that carries a selectable marker (IRES-Hygro^{R}). The TR binds tetracycline resulting in a drastic decrease in DNA binding affinity. Hence, U6TO-promoter activity is repressed in TR expressing cells; U6TO-expression is reinstated by derepressing the TR with tetracycline added to the cell culture medium.

Construction of Specific Clones

To construct a specific hp-RNA expressing vector first pick an siRNA sequence between 24-29 bases starting with a G (the preferred initiation base for PolIII). Next add a 4 to 8 base loop sequence followed by the antiparallel siRNA sequence. This sequence is inserted into a PCR primer:

5′-CCAAACGCGTAAAAA-sense-Loop-antisense-GGTGTTTCGTCCTTTCCACAAG

For example, the following primer was used to construct an EFS-U6TO vector that expresses an hp-RNA (24 bp siRNA with an 8 ntd. loop) directed against the β3-integrin (EFS-U6TO-G24):

5′-CCAAACGCGTAAAAAGAACTATTAGAGCTGCCTGTGCCTCAAGCTT
CAGGCACAGGCAGCTCTAATAGTTCGGTGTTTCGTCCTTTCCACAAG

This hp-RNA primer is used together with a second primer (US-F: 5′-CAGAGGAACAGGTCGACCAAGGTC) to PCR a portion of the U6TO promoter from the base vector. The resultant ˜350 bp fragment is digested with MluI and SalI and cloned into the same cut EFS-U6TO vector. Clones are sequence verified using the U6-F primer (5′-GGACTATCATATGCTTAC).

Generation of Retroviruses

A standard protocol (Swift et al., 1999) is used to generate infectious retrovirus from PHOENIX packaging cells. Transfection efficiency is assessed by GFP fluorescence. A standard protocol is also used to infect cells. Note that the EFS-U6TO vectors have a somewhat reduced infection rate relative to the CRU5-vectors. The infection rate is monitored by GFP fluorescence.

Amendments to the Specification:

Please replace the paragraph beginning at page 2, line 32, with the following:

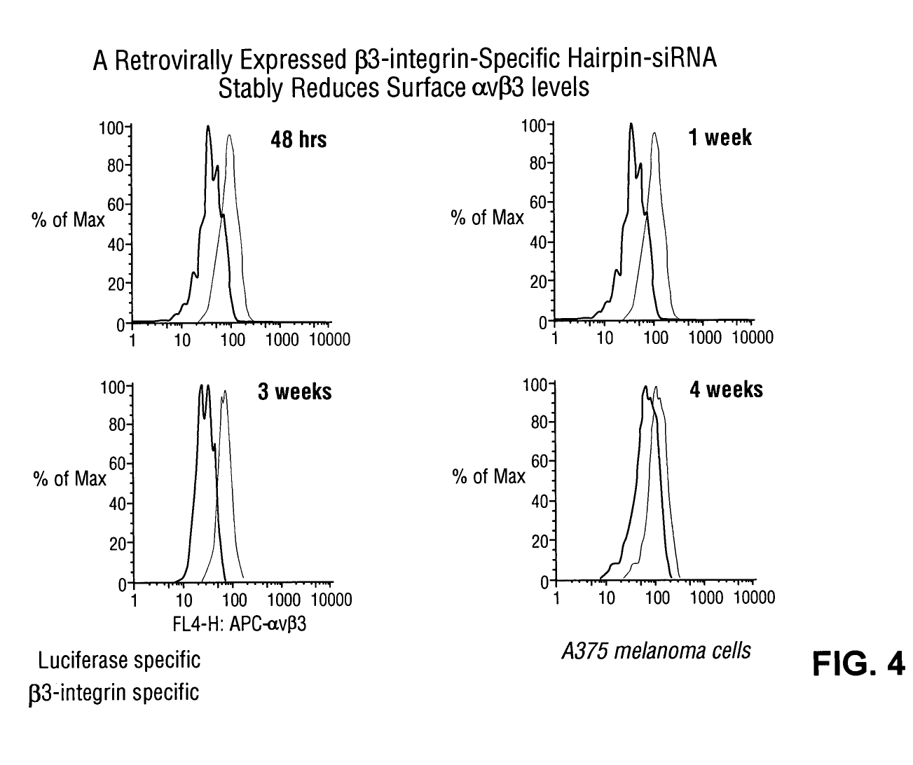

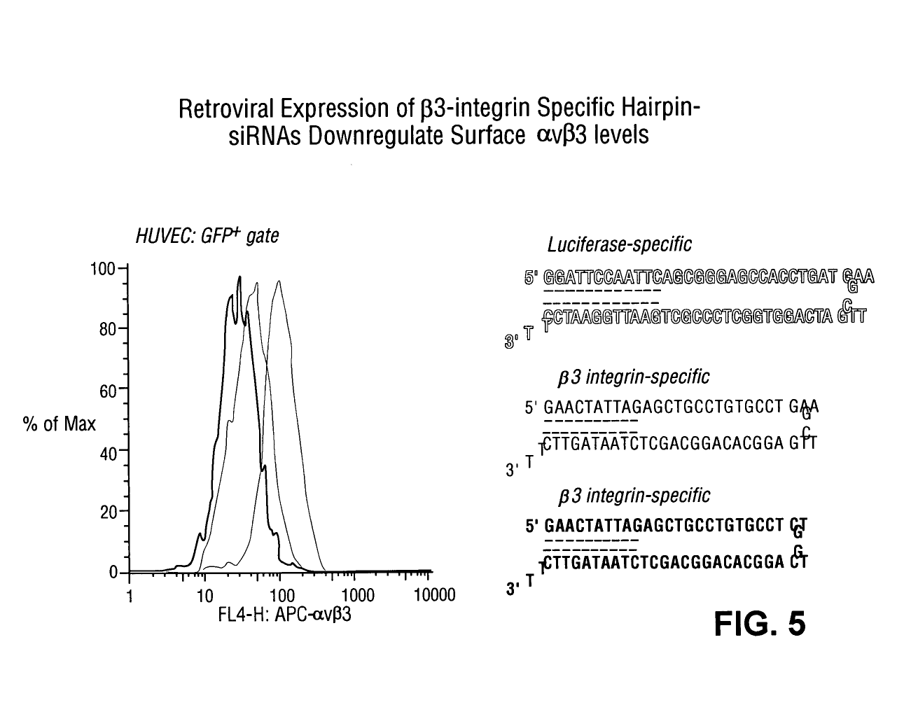

FIGS. 4 and 5 show that a retrovirally expressed β3-integrin specific hairpin siRNA stably reduces surface αv β3 levels (FIG. 5 sequences=SEQ ID NOS: 1-6).

Please replace the paragraph beginning at page 17, line 25, with the following:

To construct a specific hp-RNA expressing vector first pick an siRNA sequence between 24-29 bases starting with a G (the preferred initiation base for PolIII). Next add a 4 to 8 base loop sequence followed by the antiparallel siRNA sequence. This sequence is inserted into a PCR primer:

5′-CCAAACGCGTAAAAA-sense-Loop-antisense-GGTGTTTCGTCCTTTCCACAAG (SEQ ID NOS: 7 and 8).

Please replace the paragraph beginning at page 18, line 1, with the following:

For example, the following primer was used to construct an EFS-U6TO vector that expresses an hp-RNA (24 bp siRNA with an 8 ntd. loop) directed against the β3-integrin (EFS-U6TO-G24):

(SEQ ID NO:9)

5′-CCAAACGCGTAAAAAGAACTATTAGAGCTGCCTGTGCCTCAAGCTTC
AGGCACAGGCAGCTCTAATAGTTCGGTGTTTCGTCCTTTCCACAAG.

Please replace the paragraph beginning at page 18, line 8, with the following:

This hp-RNA primer is used together with a second primer (US-F: 5′-CAGAGGAACAGGTCGACCAAGGTC: SEQ ID NO: 10) to PCR a portion of the U6TO promoter from the base vector. The resultant ˜350 bp fragment is digested with MluI and SalI and cloned into the same cut EFS-U6TO vector. Clones are sequence verified using the U6-F primer (5′-GGACTATCATATGCTTAC; SEQ ID NO: 11).

Please insert the accompanying paper copy of the Sequence Listing, page numbers 1 to 3, at the end of the application.

We claim:

1. An expression vector comprising an expression cassette comprising, in the following sequence, a pol III promoter, a sequence encoding a first siRNA, a sequence encoding a linker RNA, a sequence encoding a second siRNA, and a termination sequence, wherein the first and the second siRNA sequences are complementary and hybridize to form a double-stranded siRNA that is about 15 to about 30 nucleotides in length.

2. The vector of claim 1, wherein the expression vector is a retroviral vector.

3. The vector of claim 2, wherein the retroviral vector is self-inactivating upon integration.

4. The vector of claim 1, wherein the expression vector is a conditional expression vector.

5. The vector of claim 4, wherein the conditional expression is conferred by a tet operator sequence overlapping the pol III promoter.

6. The vector of claim 1, comprising a marker of viral infection.

7. The vector of claim 6, wherein the marker is Renilla green fluorescent protein.

8. The vector of claim 1, wherein the siRNA is about 19 to about 28 nucleotides in length.

9. The vector of claim 1, wherein the siRNA is about 24 to about 29 nucleotides in length.

10. The vector of claim 1, wherein the linker encodes a U-turn RNA of at least about 4-8 nucleotides, and wherein the U-turn RNA forms a loop structure.

11. The vector of claim 1, wherein the linker encodes a U-turn RNA of at least about 5-6 nucleotides, and wherein the U-turn RNA forms a loop structure.

12. The vector of claim 1, wherein the pol III promoter comprises a U6 RNA promoter.

13. The vector of claim 1, wherein the sequences encoding the first and the second siRNAs are complementary to a mammalian gene.

14. The vector of claim 13, wherein the mammalian gene is associated with lymphocyte activation, angiogenesis, apoptosis, cellular proliferation, mast cell degranulation, viral replication, and viral translation.

15. The vector of claim 1, wherein the expression vector is a retroviral, conditional expression vector as depicted in FIG. 3.

16. A library comprising expression vector according to claim 1.

17. A library of expression vectors encoding double stranded siRNA molecules, each expression vector comprising an expression cassette comprising, in the following sequence, a poll III promoter, a sequence encoding a first siRNA, a sequence encoding a linker RNA, a sequence encoding a second siRNA, and a termination sequence, wherein the first and the second siRNA sequences are complementary and hybridize to form a double-stranded siRNA.

18. The library of claim 17, wherein the expression vector is a retroviral vector.

19. The library of claim 18, wherein the retroviral vector is self-inactivating upon integration.

20. The library of claim 17,wherein the expression vector is a conditional expression vector.

21. The library of claim 20, wherein the conditional expression is conferred by a tet operator sequence overlapping the pol III promoter.

22. The library of claim 17,comprising a marker of viral infection.

23. The library of claim 22, wherein the marker is Renilla green fluorescent protein.

24. The library of claim 17, wherein the library encodes randomized siRNA molecules.

25. The library of claim 17, wherein the siRNA molecules hybridize under stringent hybridization conditions to a cellular RNA population or a corresponding cDNA population.

26. The library of claim 17, wherein the siRNA is about 19 to about 28 nucleotides in length.

27. The library of claim 17, wherein the siRNA is about 24 to about 29 nucleotides in length.

28. The library of claim 17, wherein the linker encodes a U-turn RNA of at least about 4-8 nucleotides, and wherein the U-turn RNA forms a loop structure.

29. The library of claim 17, wherein the linker encodes a U-turn RNA of at least about 5-6 nucleotides, and wherein the U-turn RNA forms a loop structure.

30. The library of claim 17, wherein the pol III promoter comprises a U6 RNA promoter.

31. The library of claim 17, wherein the sequences encoding the first and the second siRNAs are complementary to a mammalian gene.

32. The library of claim 17, wherein the expression vector is a retroviral, conditional expression vector as depicted in FIG. 3.

33. A method of reducing expression of a target transcript in a cell, the method comprising the step of expressing in a cell comprising the target transcript an expression cassette of claim 1, thereby reducing expression of the target transcript.

34. The method of claim 33, wherein the target transcript is endogenously expressed.

35. The method of claim 33, wherein the target transcript is recombinantly expressed.

36. The method of claim 33, wherein the target transcript encodes a protein domain.

37. A method of identifying a gene or genes associated with a selected phenotype, the method comprising the steps of:
(i) transducing cells with the library of expression vectors encoding randomized, double-stranded siRNAs of claim 24;
(ii) assaying the cells for the selected phenotype; and
(iii) identifying, in cells that exhibit the selected phenotype, the gene or genes whose expression is modulated by expression of a randomized siRNA, wherein the gene so identified is associated with the selected phenotype.

38. The method of claim 37, wherein the phenotype is selected from the group consisting of lymphocyte activation, angiogenesis, apoptosis, cellular proliferation, mast cell degranulation, viral replication, and viral translation.
